SEQUENCE LISTING (44 sequences)

SEQ ID NO: 1 - DNA, 22 bp, synthetic construct
  misc_feature 1..22: note = synthesized
  source 1..22: mol_type = other DNA
tgaccggagaagtttcgagaac

SEQ ID NO: 2 - DNA, 21 bp, synthetic construct
  misc_feature 1..21: note = synthesized
  source 1..21: mol_type = other DNA
atcggtccagagcatcagctt

SEQ ID NO: 3 - DNA, 23 bp, synthetic construct
  misc_feature 1..23: note = synthesized
  source 1..23: mol_type = other DNA
agagtggtgtctcattcgacaaa

SEQ ID NO: 4 - DNA, 19 bp, synthetic construct
  misc_feature 1..19: note = synthesized
  source 1..19: mol_type = other DNA
gcacagctcgatgcatttc

SEQ ID NO: 5 - DNA, 21 bp, synthetic construct
  misc_feature 1..21: note = synthesized
  source 1..21: mol_type = other DNA
tgaagcaagagcgttggctaa

SEQ ID NO: 6 - DNA, 20 bp, synthetic construct
  misc_feature 1..20: note = synthesized
  source 1..20: mol_type = other DNA
cccttgttccagcgcatatc

SEQ ID NO: 7 - DNA, 21 bp, synthetic construct
  misc_feature 1..21: note = synthesized
  source 1..21: mol_type = other DNA
cgaagtgattcaggccaagtc

SEQ ID NO: 8 - DNA, 21 bp, synthetic construct
  misc_feature 1..21: note = synthesized
  source 1..21: mol_type = other DNA
cgaagtgattcaggccaagtc

SEQ ID NO: 9 - DNA, 20 bp, synthetic construct
  misc_feature 1..20: note = synthesized
  source 1..20: mol_type = other DNA
tggtgaccgtagccgtgtac

SEQ ID NO: 10 - DNA, 21 bp, synthetic construct
  misc_feature 1..21: note = synthesized
  source 1..21: mol_type = other DNA
tcagccagccgacataaaaga

SEQ ID NO: 11 - DNA, 24 bp, synthetic construct
  misc_feature 1..24: note = synthesized
  source 1..24: mol_type = other DNA
agatcaacttcaaggtcggagaag

SEQ ID NO: 12 - DNA, 19 bp, synthetic construct
  misc_feature 1..19: note = synthesized
  source 1..19: mol_type = other DNA
cgcgggtccagtaggtctt

SEQ ID NO: 13 - DNA, 22 bp, synthetic construct
  misc_feature 1..22: note = synthesized
  source 1..22: mol_type = other DNA
gtccagggtacaggtgtggttt

SEQ ID NO: 14 - DNA, 23 bp, synthetic construct
  misc_feature 1..23: note = synthesized
  source 1..23: mol_type = other DNA
ccacttgctccactctctgaact

SEQ ID NO: 15 - DNA, 20 bp, synthetic construct
  misc_feature 1..20: note = synthesized
  source 1..20: mol_type = other DNA
cgcctggagaaagctgctaa

SEQ ID NO: 16 - DNA, 20 bp, synthetic construct
  misc_feature 1..20: note = synthesized
  source 1..20: mol_type = other DNA
ccccagcatcgaaggtagag

SEQ ID NO: 17 - DNA, 19 bp, synthetic construct
  misc_feature 1..19: note = synthesized
  source 1..19: mol_type = other DNA
cgcggtctgtgaggtcact

SEQ ID NO: 18 - DNA, 19 bp, synthetic construct
  misc_feature 1..19: note = synthesized
  source 1..19: mol_type = other DNA
cgcggtctgtgaggtcact

SEQ ID NO: 19 - DNA, 19 bp, synthetic construct
  misc_feature 1..19: note = synthesized
  source 1..19: mol_type = other DNA
cgcggtctgtgaggtcact

SEQ ID NO: 20 - DNA, 19 bp, synthetic construct
  misc_feature 1..19: note = synthesized
  source 1..19: mol_type = other DNA
cgcggtctgtgaggtcact

SEQ ID NO: 21 - DNA, 18 bp, synthetic construct
  misc_feature 1..18: note = synthesized
  source 1..18: mol_type = other DNA
ggacatcgcacgcaggtt

SEQ ID NO: 22 - DNA, 21 bp, synthetic construct
  misc_feature 1..21: note = synthesized
  source 1..21: mol_type = other DNA
cgtcattgagagacccgaaag

SEQ ID NO: 23 - DNA, 19 bp, synthetic construct
  misc_feature 1..19: note = synthesized
  source 1..19: mol_type = other DNA
aaccctgctcggtcctttg

SEQ ID NO: 24 - DNA, 19 bp, synthetic construct
  misc_feature 1..19: note = synthesized
  source 1..19: mol_type = other DNA
cgcggtctgtgaggtcact

SEQ ID NO: 25 - DNA, 22 bp, synthetic construct
  misc_feature 1..22: note = synthesized
  source 1..22: mol_type = other DNA
tccatgcttaagagcgctagct

SEQ ID NO: 26 - DNA, 27 bp, synthetic construct
  misc_feature 1..27: note = synthesized
  source 1..27: mol_type = other DNA
gtatcttgtctttcatcctgatttcca

SEQ ID NO: 27 - DNA, 21 bp, synthetic construct
  misc_feature 1..21: note = synthesized
  source 1..21: mol_type = other DNA
ccggccacctatacctacgat

SEQ ID NO: 28 - DNA, 24 bp, synthetic construct
  misc_feature 1..24: note = synthesized
  source 1..24: mol_type = other DNA
tcctgaaaaggaggagtggatgta

SEQ ID NO: 29 - DNA, 18 bp, synthetic construct
  misc_feature 1..18: note = synthesized
  source 1..18: mol_type = other DNA
ccgggagaggacgacgtt

SEQ ID NO: 30 - DNA, 18 bp, synthetic construct
  misc_feature 1..18: note = synthesized
  source 1..18: mol_type = other DNA
ccgggagaggacgacgtt

SEQ ID NO: 31 - DNA, 18 bp, synthetic construct
  misc_feature 1..18: note = synthesized
  source 1..18: mol_type = other DNA
ccgggagaggacgacgtt

SEQ ID NO: 32 - DNA, 20 bp, synthetic construct
  misc_feature 1..20: note = synthesized
  source 1..20: mol_type = other DNA
gaaactgccgcccaactaga

SEQ ID NO: 33 - DNA, 20 bp, synthetic construct
  misc_feature 1..20: note = synthesized
  source 1..20: mol_type = other DNA
tccgaaagttgcaacgagaa

SEQ ID NO: 34 - DNA, 21 bp, synthetic construct
  misc_feature 1..21: note = synthesized
  source 1..21: mol_type = other DNA
ccgtggatggaataagggaaa

SEQ ID NO: 35 - DNA, 23 bp, synthetic construct
  misc_feature 1..23: note = synthesized
  source 1..23: mol_type = other DNA
catggactgtggtcatgagtcct

SEQ ID NO: 36 - DNA, 23 bp, synthetic construct
  misc_feature 1..23: note = synthesized
  source 1..23: mol_type = other DNA
catggactgtggtcatgagtcct

SEQ ID NO: 37 - DNA, 20 bp, synthetic construct
  misc_feature 1..20: note = synthesized
  source 1..20: mol_type = other DNA
acacccaggcagagaatgct

SEQ ID NO: 38 - DNA, 21 bp, synthetic construct
  misc_feature 1..21: note = synthesized
  source 1..21: mol_type = other DNA
cgaagactgggctctccttgt

SEQ ID NO: 39 - DNA, 18 bp, synthetic construct
  misc_feature 1..18: note = synthesized
  source 1..18: mol_type = other DNA
gtgtgccccacggagatc

SEQ ID NO: 40 - DNA, 20 bp, synthetic construct
  misc_feature 1..20: note = synthesized
  source 1..20: mol_type = other DNA
catgggtcccagtcctcctt

SEQ ID NO: 41 - DNA, 21 bp, synthetic construct
  misc_feature 1..21: note = synthesized
  source 1..21: mol_type = other DNA
tgcctggagcaaggatatcaa

SEQ ID NO: 42 - DNA, 20 bp, synthetic construct
  misc_feature 1..20: note = synthesized
  source 1..20: mol_type = other DNA
gtcacaggcatgcccttttc

SEQ ID NO: 43 - DNA, 21 bp, synthetic construct
  misc_feature 1..21: note = synthesized
  source 1..21: mol_type = other DNA
catgttcgtcatgggtgtgaa

SEQ ID NO: 44 - DNA, 23 bp, synthetic construct
  misc_feature 1..23: note = synthesized
  source 1..23: mol_type = other DNA
catggactgtggtcatgagtcct

(19) United States
(12) Patent Application Publication
(10) Pub. No.: US 2024/0226177 A1
(43) Pub. Date: Jul. 11, 2024

(54) RETINAL PIGMENT EPITHELIAL CELL TRANSPLANTATION FOR THE TREATMENT OF CORNEAL ENDOTHELIAL DYSFUNCTION

(71) Applicant: EYE INSTITUTE OF SHANDONG FIRST MEDICAL UNIVERSITY, Qingdao (CN)

(72) Inventors: Weiyun Shi, Qingdao (CN); Qingjun Zhou, Qingdao (CN); Zongyi Li, Qingdao (CN); Chunxiao Dong, Qingdao (CN); Haoyun Duan, Qingdao (CN)

(21) Appl. No.: 18/592,478

(22) Filed: Feb. 29, 2024

Related U.S. Application Data
(63) Continuation-in-part of application No. 18/352,690, filed on Jul. 14, 2023.
Application No. 18/352,690 is a continuation of application No. PCT/CN2022/105465, filed on Jul. 13, 2022.

(51) Int. Cl. A61K 35/30; A61K 31/4406; A61K 31/4439; A61P 27/02; C12N 5/079

(52) U.S. Cl. CPC A61K 35/30; A61K 31/4406; A61K 31/4439; A61P 27/02; C12N 5/0621

(57) ABSTRACT

Retinal pigment epithelial cells can be used for replacing corneal endothelial cells, preventing and treating diseases or symptoms such as corneal endothelial functional decompensation. The retinal pigment epithelial cell transplantation, e.g., to the anterior chamber of an eye of the subject, can restore corneal transparency, reduce corneal thickness, reconstruct corneal endothelial barrier function, effectively treat corneal endothelial functional decompensation and corneal endothelial dysfunction.

### INCORPORATION OF SEQUENCE LISTING

This application contains a sequence listing submitted in Computer Readable Form (CRF). The CFR file contains the sequence listing entitled “PA7410004CIP-SequenceListing.xml”, which was created on Feb. 29, 2024, and is 56,274 bytes in size. The information in the sequence listing is incorporated herein by reference in its entirety.

### TECHNICAL FIELD

The present invention belongs to the field of medicine, and relates to an application of retinal pigment epithelial cells in relieving or treating corneal endothelial functional decompensation.

### BACKGROUND

The cornea is a transparent tissue located on the anterior of the eyeball, and is divided into five layers, which sequentially comprises epithelial cell layer, bowman membrane, stromal layer, descemet's membrane, and endothelial cell layer from front to back. The highly transparent and optical properties of the cornea are one of the prerequisites that normally exert physiological functions, while corneal endothelial cells play an important role in maintaining the normal physiological function of the cornea. Corneal endothelial cells are single-layer cells in corneal inner layer, which forms a physical barrier between descemet's membrane and aqueous humor, adjusts the concentration and moisture of ions in the cornea by the ion “pump” function to maintain the semi-dehydration state of the cornea, and ensures the normal thickness and transparency of the cornea. Once the function of corncal endothelial cells is disordered, it often leads to corneal edema, causing partial or even complete corneal blindness.

Normal human corneal endothelial cells have extremely limited proliferation capacity in vivo. Endothelial cell damage and loss caused by trauma, inflammation, cataract surgery, etc., can only be filled by the enlargement and migration of surrounding cells. When the density of human corneal endothelial cells drops to its physiological critical value (about 400-500 cells/mm²), corneal edema occurs and vision loss in severe cases occurs. At present, there are about 4 million patients with corneal blindness in China, including nearly 1 million patients with endothelial blindness. Corneal transplantation is the only clinical therapeutic strategy for corneal endothelial decompensation. Due to the lack of corneal donors in China, only less than 10000 patients regain their sight through corneal transplantation every year, which is far from covering all clinical needs. In order to solve the problem of the corneal donor shortage, the current main corneal endothelial alternative seed cell research comprises cultured adult stem cells such as human corneal endothelial cells and skin progenitor cells, and corneal endothelial cells derived from human embryonic stem cells (hESCs) and human induced pluripotent stem cells (hiPSCs).

Although the transplantation of primary corneal endothelial cells, adult stem cells, or pluripotent stem cells-derived cells can improve corneal endothelial function, the effect is limited. Up to now, there are no ideal corneal endothelial alternative seed cells to achieve long-term corneal transparency and wide clinical application. The team of Japanese scientist Professor Kinoshita transplanted human corneal endothelial cells by anterior chamber injection to clinically treat 11 patients with corncal endothelial dystrophy, and they restored corneal transparency, but the results of 5-year follow-up showed that the corneal endothelium of some patients reappeared pathological “guttac” structure. Cultured human corneal endothelial cells still rely on high-quality donor cornea, and adult corneal endothelial seed cells cannot be expanded in vitro in large quantities, so the source and number of these cells is limited; Adult stem cell-derived alternative cells, such as skin progenitor cells, have poor purity, difficult industrial preparation and limited therapeutic effect; hESC/hiPSC has unlimited proliferative ability and pluripotency, it has been reported that hESC/hiPSC differentiated into neural crest, corneal endothelial precursor and mature corneal endothelioid cells, some experiments have proved that corneal endothelial precursor cells and mature corneal endothelioid cells can be applied to animal models to restore corneal transparency, however, there is currently no standardized method for directed differentiation of hESC/hiPSC to corneal endothelial cells, the long-term efficacy and safety of hESC/hiPSC-derived corneal endothelial cells in vivo remains to be studied. Therefore, to find the ideal corneal endothelial alternative seed cells is still an urgent problem to be solved in the field of corneal endothelial treatment.

### SUMMARY OF THE INVENTION

The inventors find that although retinal pigment epithelial cells are greatly different from corneal endothelial cells in terms of tissue differentiation source, anatomical location, and somatic tissue cell function, they have a homologous regular hexagonal morphology and express tight junction proteins, suggesting that retinal pigment epithelial cells have the possibility of providing barrier function in substitute of corneal endothelial cells and treating corneal endothelial functional decompensation. In order to solve the shortcomings of the prior art, the object of the present invention is to provide corneal endothelial alternative cells; the further objective is to select hESC/hiPSC-derived retinal pigment epithelial cells as seed cells, providing a seed cell source that could be supplied indefinitely and used clinically safely; more further purpose is to improve the function of transplanted cells, optimize the preparation process of cell suspension, and ensure the normal function of transplanted cells.

In order to achieve the above objectives, the present invention provides a method for relieving or treating a corneal thickness abnormality, corneal transparency decline, a corneal edema, a corneal endothelial injury, a corneal endothelial lesion, vision decline, vision loss, eye dryness, or eye pain in a subject, which comprises administering to the subject retinal pigment epithelial cells.

In a preferred embodiment of the present invention, the retinal pigment epithelial cells are administered in the form of a composition comprising a cell medium solvent, wherein the ratio of the retinal pigment epithelial cells to the DMEM low-sugar culture medium is 3×10⁵-1.2×10⁶: 200-300 microliters.

In a preferred embodiment of the present invention, the cell medium solvent is DMEM low-sugar culture medium; and the ratio of the retinal pigment epithelial cells to the DMEM low-sugar culture medium is 5×10⁵-1×10⁶: 200-300 microliters.

In a preferred embodiment of the present invention, the method further comprises administering to the subject one or more specific inhibitors selected from Y27632, nicotinamide, and TGF-β inhibitor SB431542.

In a preferred embodiment of the present invention, the method further comprises administering to the subject 5-100 μM Y27632.

In a preferred embodiment of the present invention, the subject suffers from corneal endothelial function decompensation or corneal endothelial cell dysfunction.

In a preferred embodiment of the present invention, the retinal pigment epithelial cells are administered to the subject's eye. Especially, into the anterior chamber of the subject's eye.

In a preferred embodiment of the present invention, the retinal pigment epithelial cells are obtained by differentiating stem cells selected from human embryonic stem cells, human-induced pluripotent stem cells or primary culture of human or rabbit retinal pigment epithelial cells.

In a preferred embodiment of the present invention, the pigment-producing gene Tyrosinase in the cells are knocked out.

In a preferred embodiment of the present invention, the pigment epithelial cells express corneal endothelial functional markers ZO1 and ATP1A1.

In a preferred embodiment of the present invention, the retinal pigment epithelial cells were obtained by differentiating stem cells selected from human embryonic stem cells or human-induced pluripotent stem cells and the retinal pigment epithelial cells are injected into the anterior chamber of the subject's eye.

The present invention also provides a method for relieving or treating a corneal edema, a corneal thickness abnormality, or corneal transparency decline in a subject, which comprises administering to the subject retinal pigment epithelial cells and a DMEM low-sugar culture medium, wherein the ratio of the retinal pigment epithelial cells to the DMEM low-sugar culture medium is 3×10⁵-1.2×10⁶: 200-300 microliters.

In a preferred embodiment of the present invention, the ratio of the retinal pigment epithelial cells to the DMEM low-sugar culture medium is 5×10⁵-1×10⁶: 200-300 microliters.

In a preferred embodiment of the present invention, the method further comprises administering to the subject one or more specific inhibitors selected from Y27632, nicotinamide, and TGF-β inhibitor SB43 1 542.

The retinal pigment epithelial cells provided by the present invention can be administered in any convenient dosage form, and the preferred dosage form comprises injection, cell sheet or kit. Regardless of the dosage form, retinal pigment epithelial cells are administered to the anterior chamber of the individual's eyeballs.

The beneficial technical effects of the present invention are as follows:

According to the invention, retinal pigment epithelial cells are used as alternative seed cells of corneal endothelium for the first time; the cell suspension and the preparation method provided by the invention can effectively replace corneal endothelial function to recover corneal transparency and corneal thickness while ensuring cell viability. In addition, the seed cells for replacing corneal endothelium provided by the present invention can be obtained by inducing differentiation from hESC/hiPSCs, and are infinitely supplied, and the application security thereof has been reported in existing clinical experiments. According to the preparation method and the transplantation method provided by the invention, highly specialized equipment, reagents or skills are not needed, so that researchers and clinical personnel can operate conveniently, and therefore, the method has wide application values and positive social benefits.

### BRIEF DESCRIPTION OF THE DRAWINGS

FIGS. 1A, 1B, 1C, 1D and 1E Similarities and differences between non-pigmented rabbit RPE cells and CECs in situ. FIG. 1A: Schematic diagram of the morphology and location of RPE cells and CECs in rabbits created with BioRender.com. FIG. 1B: qRT-PCR analysis of OTX2, CRELBP, BEST1, MITF, RPE65, ATP1A1, and TJP1 between Rb-RPE and Rb-CEC. Quantification represented the levels of relative mRNA expressions normalized to GAPDH. FIG. 1C: Representative immunofluorescence images of Rb-RPE and Rb-CEC in situ were collected, including ZO1 (green), ATP1A1 (red) and retinal pigment epithelial markers MITF (green), and RPE65 (red). Nuclei were stained with DAPI (blue) (scale bar: 50 μm). FIG. 1D: Cell density, hexagonality, and coefficient of variation analysis were based on ZO1 immunostaining. FIG. 1E: Scanning electron microscope showed a regular hexagonal shape in both Rb-RPE and Rb-CEC, which were well formed and with distinct cell boundaries (scale bar: 10 μm) (Upper). Transmission electron microscope showed that both types of cells were attached to their respective basement membrane, named BM and DM, by hemidesmosomal junctions ( [symbol] ) (scale bar: 500 nm) (Middle). Adjacent cells were joined with numerous well-developed tight junctions ( [symbol] ) (scale bar: 500 nm) (Lower). Data are mean ±SEM. All results were obtained from three independent experiments. Significance (*P<0.05, ** P<0.01 and ns: nonsignificant) relative to Rb-CEC. BM: Bruch's membrane. DM: Descemet's membrane. Rb-CEC: rabbit CECs. Rb-RPE: rabbit RPE cells.

FIGS. 2A, 2B, 2C and 2D Confluent cultures of primary RPE cells and primary CECs taken from non-pigmented rabbit eyeball. FIG. 2A: Cell morphology of Rb-CEC and Rb-RPE in Example 1 were assessed with an inverted phase-contrast microscope (scale bar: 100 μm). FIG. 2B: Representative immunofluorescence staining images of corneal endothelial markers ZO1 (green), and ATP1A1 (red) and retinal pigment epithelial markers MITF (green), and RPE65 (red) in Rb-RPE and Rb-CEC. Nuclei were stained with DAPI (blue) (scale bar: 50 μm). FIG. 2C: using HRP tracer at 5 min, 10 min, 15 min, and 30 min, respectively. FIG. 2D: The Na+, K+-ATPase activities of Rb-RPE and Rb-CEC at passage 2. Data are mean ±SEM. All results were obtained from three independent experiments. Significance (** P<0.01, ns: nonsignificant) relative to Rb-CEC. Rb-CEC: rabbit CECs. Rb-RPE: rabbit RPE cells.

FIGS. 3A, 3B, 3C, 3D, 3E and 3F show the repair effect of rabbit primary retinal pigment epithelial cells on corneal endothelial dysfunction. FIGS. 3A and 3B: Corneal transparency of rabbits injected with non-pigmented CECS and RPE cells were observed by a slit lamp microscope at days 1, 3, 7, and 14, respectively; FIG. 3C: OCT image-based central corneal thickness analysis; FIG. 3D: IOP was measured by tonometer at days 1, 3, 7, and 14, respectively; FIG. 3E: Immunofluorescence staining of ZO1 (green) and RPE65(green) in the central area of corneas transplanted with non-pigmented Rb-RPE and Rb-CEC at day 14 postoperatively. Nuclei were stained with DAPI (blue) (scale bar: 50 μm); FIG. 3F: Immunofluorescent stainings of IgG isotype controls. Transplanted primary non-pigmented rabbit CECs and RPE cells were stained with mouse IgG isotype controls (scale bar: 50 μm). Data are mean ±SEM. In vivo experiments were performed using three independent animals per group. Significance (ns: nonsignificant) relative to Rb-CEC. IOP: intraocular pressure. OCT: optical coherence tomography. Rb-CEC: rabbit CECs. Rb-RPE: rabbit RPE cells.

FIGS. 4A, 4B, 4C and 4D Characterization and therapeutic effects of primary pigmented RPE cells in vitro. FIG. 4A: Cell morphology of primary pigmented RPE cells at passages 0-2 (scale bar: 100 μm). FIG. 4B: Representative images of functional markers ZO1 (green), ATP1A1 (red) and RPE-related markers MITF (green), and RPE65 (red) in pigmented RPE cells cultured in vitro were recorded by immunofluorescence staining. Nuclei were stained with DAPI (blue) (scale bar: 50 μm). FIG. 4C: Corneal transparency was observed on days 1, 3, 7, and 14 postoperatively. FIG. 4D: Transplanted pigmented RPE cells were stained with ZO1 (green) and RPE65 (green) at day 14 after surgery. Nuclei were stained with DAPI (blue) (scale bar: 50 μm).

FIGS. 5A and 5B Characterization of hESC-derived RPE cells in vitro. FIG. 5A: Cell morphology of hESCs (scale bar: 200 μm) and hESC-derived RPE cells (scale bar: 100 μm), respectively. FIG. 5B: Representative immunostaining images of hESC-derived RPE cells (scale bar: 20 μm).

FIGS. 6A, 6B, 6C, 6D, 6E and 6F show therapeutic effects of hESC-derived RPE cells for the treatment of corneal endothelial dysfunction. FIG. 6A: Corneas with transplanted hESC-derived retinal pigment epithelial cells achieved a rapid improvement of corneal clarity and remained stable from day 7 after operation; FIG. 6B: Corneas with transplanted hiPSC-derived retinal pigment epithelial cells achieved a rapid improvement of corneal clarity and remained stable from day 7 after operation;FIG. 6C: Immunofluorescence staining images of α-SMA (green) and Vimentin (green) in the transplanted area 7 days after surgery. Nuclei were stained with DAPI (blue) (scale bar: 25 μm); FIG. 6D: ZO1 (green), ATP1A1 (green) and MITF (red) were stained in the central area of the cornea 14 days after surgery. The transplanted cells were stained with human specific antibody human nuclei (HuNu) (red). Nuclei were stained with DAPI (blue) (scale bar: 50 μm);FIG. 6E: Immunofluorescence staining images of actin (green) and human cell-surface determinant TRA-1-85 (red) at day 14 postoperatively. Nuclei were stained with DAPI (blue); FIG. 6F: qRT-PCR analysis of CEC related genes, RPE related genes, and antioxidant genes between cultured hESC-derived RPE cells in vitro and transplanted hESC-derived RPE cells. Quantification represented the levels of relative mRNA expressions normalized to GAPDH.

FIG. 7 is representative images of corneal transparency and central corneal thickness at day 1, 7, 14 and 30 postoperatively. Tyro KO-iRPE cells: hESC-derived RPE cells. iRPE: normal hESC-derived RPE cells.

### DETAILED DESCRIPTION OF THE EMBODIMENTS

The present invention is further illustrated by the following embodiments explaining the present invention, the following embodiments are only used to illustrate the present invention and should not be regarded as limiting the scope of the present invention. Unless otherwise indicated, the technical and scientific terms used herein are generally understood by those of ordinary skill in the art to which the invention belongs. If the specific conditions are not indicated in the embodiment, the conditions recommended by the manufacturer shall be carried out in accordance with the general conditions or the conditions recommended by the manufacturer. The reagents or instruments used are conventional products that are commercially available if the manufacturer is not identified. Animals Sources

In the present invention, non-pigmented and pigmented New Zealand white rabbits and grey rabbits were purchased from Jinan Xilingjiao Breeding Center (Shangdong, China) Non-pigmented rabbits (3.5 kg, six months old) were used to establish the model of corneal endothelial dysfunction, while three-months-old rabbits were used for primary rabbit RPE cells and CECs culture.

The hESC cell line H1 was donated by Professor Yin Zhengqin's laboratory; The hiPSC cell line DY0100 was purchased from the Chinese Academy of Sciences Cell Bank/Stem Cell Bank; Tryosinase-specific knockout hESC H1 cell line: Tryosinase-specific knockout virus were purchased from Shanghai GK Gene Medical Technology Co., Ltd. and the Tryosinase-specific knockout hESC H1 cell line was prepared according to its instruction;

### General Methods

Quantitative Real-Time Reverse Transcription Polymerase Chain Reaction (qRT-PCR)

The relative differential mRNA expressions of rabbit RPE cells and CECs were monitored by qRT-PCR analysis. Total RNA was extracted using the MiniBEST Universal RNA Extraction Kit (TakaRa, Tokyo, Japan). Next, complementary DNA (cDNA) was synthesized by HiScript III RT SuperMix for qPCR (Vazyme, Nanjing, China), and qRT-PCR was performed using SYBR Green qPCR Master Mix (Vazyme) according to the manufacturer's protocol. The quantified data were analyzed and normalized to glyceraldehyde-3-phosphate dehydrogenase (GAPDH). The primers used in the qRT-PCR were listed in Table 1.

TABLE 1
Primer sequences for quantitative reverse transcription transcription-polymerase
chain reaction

| Gene | | | | |
|---|---|---|---|---|
| name | Forward | | Reverse | |
| Rabbit | TGACCGGAGAAGTTTCGAGAAC | SEQ | ATCGGTCCAGAGCATCAGCTT | SEQ |
| TJP1 | | ID | | ID |
| | | NO: | | NO: |
| | | 1 | | 2 |
| Rabbit | AGAGTGGTGTCTCATTCGACAAA | SEQ | GCACAGCTCGATGCATTTC | SEQ |
| ATP1A1 | | ID | | ID |
| | | NO: | | NO: |
| | | 3 | | 4 |
| Rabbit | TGAAGCAAGAGCGTTGGCTAA | SEQ | CCCTTGTTCCAGCGCATATC | SEQ |
| MITF | | ID | | ID |
| | | NO: | | NO: |
| | | 5 | | 6 |
| Rabbit | CGAAGTGATTCAGGCCAAGTC | SEQ | CGAAGTGATTCAGGCCAAGTC | SEQ |
| RPE65 | | ID | | ID |
| | | NO: | | NO: |
| | | 7 | | 8 |
| Rabbit | TGGTGACCGTAGCCGTGTAC | SEQ | TCAGCCAGCCGACATAAAAGA | SEQ |
| BEST1 | | ID | | ID |
| | | NO: | | NO: |
| | | 9 | | 10 |
| Rabbit | AGATCAACTTCAAGGTCGGAGAAG | SEQ | CGCGGGTCCAGTAGGTCTT | SEQ |
| CRELBP | | ID | | ID |
| | | NO: | | NO: |
| | | 11 | | 12 |
| Rabbit | GTCCAGGGTACAGGTGTGGTTT | SEQ | CCACTTGCTCCACTCTCTGAACT | SEQ |
| OTX2 | | ID | | ID |
| | | NO: | | NO: |
| | | 13 | | 14 |
| Rabbit | CGCCTGGAGAAAGCTGCTAA | SEQ | CCCCAGCATCGAAGGTAGAG | SEQ |
| GAPDH | | ID | | ID |
| | | NO: | | NO: |
| | | 15 | | 16 |
| Human | CGCGGTCTGTGAGGTCACT | SEQ | CGCGGTCTGTGAGGTCACT | SEQ |
| CD200 | | ID | | ID |
| | | NO: | | NO: |
| | | 17 | | 18 |
| Human | CGCGGTCTGTGAGGTCACT | SEQ | CGCGGTCTGTGAGGTCACT | SEQ |
| S100A4 | | ID | | ID |
| | | NO: | | NO: |
| | | 19 | | 20 |
| Human | GGACATCGCACGCAGGTT | SEQ | CGTCATTGAGAGACCCGAAAG | SEQ |
| SLC4A11 | | ID | | ID |
| | | NO: | | NO: |
| | | 21 | | 22 |
| Human | AACCCTGCTCGGTCCTTTG | SEQ | CGCGGTCTGTGAGGTCACT | SEQ |
| AQP1 | | ID | | ID |
| | | NO: | | NO: |
| | | 23 | | 24 |
| Human | TCCATGCTTAAGAGCGCTAGCT | SEQ | GTATCTTGTCTTTCATCCTGATTTCCA | ID |
| TCF8 | | ID | | SEQ |
| | | NO: | | NO: |
| | | 25 | | 26 |
| Human | CCGGCCACCTATACCTACGAT | SEQ | TCCTGAAAAGGAGGAGTGGATGTA | SEQ |
| COL8A2 | | ID | | ID |
| | | NO: | | NO: |
| | | 27 | | 28 |
| Human | CCGGGAGAGGACGACGTT | SEQ | CCGGGAGAGGACGACGTT | SEQ |
| OTX2 | | ID | | ID |
| | | NO: | | NO: |
| | | 29 | | 30 |
| Human | CCGGGAGAGGACGACGTT | SEQ | GAAACTGCCGCCCAACTAGA | SEQ |
| BEST1 | | ID | | ID |
| | | NO: | | NO: |
| | | 31 | | 32 |
| Human | TCCGAAAGTTGCAACGAGAA | SEQ | CCGTGGATGGAATAAGGGAAA | SEQ |
| MITF | | ID | | ID |
| | | NO: | | NO: |
| | | 33 | | 34 |
| Human | CATGGACTGTGGTCATGAGTCCT | SEQ | CATGGACTGTGGTCATGAGTCCT | SEQ |
| ETS1 | | ID | | ID |
| | | NO: | | NO: |
| | | 35 | | 36 |
| Human | ACACCCAGGCAGAGAATGCT | SEQ | CGAAGACTGGGCTCTCCTTGT | SEQ |
| HO1 | | ID | | ID |
| | | NO: | | NO: |
| | | 37 | | 38 |
| Human | GTGTGCCCCACGGAGATC | SEQ | CATGGGTCCCAGTCCTCCTT | SEQ |
| PRDX1 | | ID | | ID |
| | | NO: | | NO: |
| | | 39 | | 40 |
| Human | TGCCTGGAGCAAGGATATCAA | SEQ | GTCACAGGCATGCCCTTTTC | SEQ |
| PRDX6 | | ID | | ID |
| | | NO: | | NO: |
| | | 41 | | 42 |
| Human | CATGTTCGTCATGGGTGTGAA | SEQ | CATGGACTGTGGTCATGAGTCCT | SEQ |
| GAPDH | | ID | | ID |
| | | NO: | | NO: |
| | | 43 | | 44 |

### Immunofluorescence Staining

Cultured cells were fixed with 4% paraformaldehyde (PFA) (Biosharp, Anhui, China) for 10 min. After incubating in 0.3% Triton X-100 (Beyotime Biotechnology, Shanghai, China) for 5-15 min, all species of cells were reacted with 5% bovine serum albumin (BSA) (Boster Biological Technology, Wuhan, China) for 1 h to block nonspecific binding sites at room temperature and treated with primary antibodies (Table 2) at 4° C. overnight. After reacting with Alexa Fluor 488- or 594-conjugated secondary antibodies (Invitrogen, Carlsbad, California, USA) (Table 2) for 1 h at room temperature, nuclei were stained with 4,6-diamidino-2-phenylindole (DAPI) (Beyotime Biotechnology), and then fluorescence images were captured using the Echo Revolve-100-G (ECHO, San Diego, California, USA).

Normal rabbit eyeballs were obtained post-euthanasia for in situ immunofluorescence staining of retinal pigmented epithelium and corneal endothelium. Briefly, the cornea removed from the eyeball was fixed with 4% PFA for 12 min and stored at 4° C. until use. The eye cup was obtained after removing the segments of the eye, including the iris, lens, and vitreous body. After incubating with 4% PFA for 30 min, the neurosensory retina and sclera were removed while the RPE-Buch's membrane-choriocapillaris complex (RBCC) was acquired and stored at 4° C. Subsequently, in situ rabbit cornea and RBCC were permeabilized with 0.5% Triton X-100 for 5-15 min. After washing rabbit tissues with PBS three times, they were blocked in 2.5% BSA for 1 h at room temperature and incubated with primary antibodies (Table 2) overnight at 4° C. Both the rabbit cornea and RBCC were washed to remove excess primary antibodies the next day and then incubated with secondary antibodies (Table 2) for 1 h at room temperature. The nuclei were stained with DAPI and the fluorescence images were observed by laser scanning confocal microscopy (LSM 800, Zeiss, Jena, Germany).

For the immunofluorescence testing, the corneas with the transplanted cells were obtained after euthanasia and fixed with 4% PFA for 12 min. The following immunofluorescence staining procedures were consistent with those of rabbit corneal endothelium in situ.

TABLE 2
Antibodies for immunofluorescence staining

| Antibody | Supplier | Code | Dilution |
|---|---|---|---|
| Rabbit polyclonal to ZO1 | Thermo | 40-2200 | 1:100 |
| | Fisher | | |
| Rabbit monoclonal to ATP1A1 | Abcam | ab76020 | 1:150 |
| Mouse monoclonal to MITF | Abcam | ab3201 | 1:100 |
| Rabbit polyclonal to RPE65 | Abcam | ab235950 | 1:200 |
| Mouse monoclonal to α-SMA | Abcam | ab7817 | 1:100 |
| Mouse monoclonal to human nuclei | Abcam | ab191181 | 1:150 |
| Mouse monoclonal to ZO1 | Thermo | 33-9100 | 1:50 |
| | Fisher | | |
| Mouse monoclonal to ATP1A1 | Abcam | ab7671 | 1:100 |
| Mouse monoclonal to RPE65 | Thermo | MA1-16578 | 1:100 |
| | Fisher | | |
| Mouse monoclonal to Vimentin | Abcam | ab8978 | 1:100 |
| Donkey anti-mouse | Thermo | A-21202 | 1:300 |
| IgG-AF488 (H + L) | Fisher | | |
| Donkey anti-mouse | Thermo | SA5-10168 | 1:300 |
| IgG-AF594 (H + L) | Fisher | | |
| Donkey anti-rabbit | Thermo | A-21206 | 1:300 |
| IgG-AF488 (H + L) | Fisher | | |
| Donkey anti-rabbit | Thermo | SA5-10040 | 1:300 |
| IgG-AF594 (H + L) | Fisher | | |

Electron microscopy

The RBCC and rabbit corneal endothelium were examined by scanning electron microscopy and transmission electron microscopy, respectively. The RBCC and rabbit corneal endothelium were immersed and preserved in fixative (Servicebio, Wuhan, China) at 4° C. and postfixed with 1% OsO4 in 0.1 M PB (pH 7.4) for 2 h at room temperature, avoiding light. After rinsing in 0.1 M PB (pH 7.4) three times, the tissues were dehydrated in a graded ethanol (Sinaopharm Group Chemical Reagent Co. LTD, China) series. For scanning electron microscopy preparation, the tissues were dried with a critical point dryer (Quorum, UK). Specimens were attached to metallic stubs using carbon stickers and sputter-coated with gold for 30 s before examination with scanning electron microscopy (HITACHI, Japan). For transmission electron microscopy preparation, the tissues were embedded in resin (EMBed 812). Ultrathin sections (60-80 nm thin) were fished out onto cuprum grids with formvar film and were stained with 2% uranium acetate saturated alcohol solution for 8 min as well as 2.6% lead citrate for 8 min. After drying overnight at room temperature, the cuprum grids were observed under transmission electron microscopy (hitachi), and images were taken.

### Enzyme Linked Immunosorbent Assay (ELISA)

The anterior chamber humors of the hESC-RPE cell transplanted rabbits and non-injected corneal endothelial-damaged rabbits was collected on day 30 after surgery, and the concentrations of VEGF and pigment epithelium-derived factor (PEDF) (Kete, Jiangsu, China) were quantified using the indicated ELISA kits according to the manufacturer's instructions.

### Statistical Analysis

All data obtained from at least three independent experiments were expressed as the mean ±standard error of the mean for the values. A student's t-test or one-way ANOVA was used to compare the mean values between the groups using SPSS 19.0 (SPSS, Chicago, IL, USA), while P<0.05 and P<0.01 were considered as the significance thresholds.

### Example 1 Primary Culture of Rabbit Retinal Pigment Epithelium(RPE) Cells and Corneal Endothelial Cells (CECs)

Rabbits were euthanatized, and the eyeballs were stored at 4° C. for the subsequent culture of primary rabbit RPE cells and corneal CECs. As previously published (Wiencke A K, Kiilgaard J F, Nicolini J, Bundgaard M, Röpke C, Cour M L. Growth of cultured porcine retinal pigment epithelial cells. Acta Ophthalmol Scand. 2003; 81(2): 170-6.and Klettner A, Kampers M, Töbelmann D, Roider J, Dittmar M. The influence of melatonin and light on VEGF secretion in primary RPE cells. Biomolecules. 2021; 11(1):114.), primary RPE cells were isolated and cultured for subsequent study. Briefly, the eyes were immersed in saline containing 400 U/ml gentamicin sulfate (CISEN Pharmaceutical Co., Shandong, China) for at least 30 min. Next, the intact corneas were separated and stored in Dulbecco's modified Eagle's medium (DMEM) (Corning, Manassas, VA, USA) for subsequent primary CEC culture. Excess tissues, including the lens, vitreous, and retina, were removed to obtain the optic cup, in which RPE cells were still attached on the inner side. Primary RPE cells were detached by 0.25% trypsin-EDTA (Sigma-Aldrich, Missouri, USA) for 1 h at 37° C., then gently isolated from Bruch's membrane and triturated into cell suspension. RPE cells were cultured in a medium consisting of DMEM, 10% fetal bovine serum (FBS) (Gibco, Grand Island, NY, USA), 1% penicillin/streptomycin (PS) (Corning), 10 μM Y-27632 (Sigma-Aldrich), 10 μM nicotinamide (NAM) (Sigma-Aldrich), and 1 μM SB431542 (Millipore, Boston, Massachusetts, USA) at 37° C. under 5% CO₂. A total of about 1.6×10⁴ RPE cells could be obtained from one single eye of rabbits, and 8×10³ cells were seeded in one well of a 24-well plate. Cultured RPE cells were observed and checked microscopically every other day. Contaminant cells (such as fibroblasts) were removed mechanically. The cultures used for experimentation were confluent and exhibited a typical cobblestone-like morphology.

Primary rabbit CECs were cultured according to a previously reported method (Okumura N, Koizumi N, Ueno M, Sakamoto Y, Takahashi H, Tsuchiya H, et al. ROCK inhibitor converts corneal endothelial cells into a phenotype capable of regenerating in vivo endothelial tissue. Am J Pathol. 2012;181(1): 268-77.). Briefly, Descemet's membrane (DM) and corneal endothelium were separated under a microscope and cultured in DMEM supplemented with 10 μM Y-27632 overnight. Next, CECs were digested from DM with 0.6 U/mL of collagenase I (Sigma-Aldrich) for 1 h and cultured in a medium including DMEM, 10% FBS, 1%PS, 2 ng/mL human basic fibroblast growth factor (bFGF, R&D System, Minneapolis, USA), 1% insulin-transferrin-selenium (ITS, Gibco), 1 μM SB431542, and 10 μM Y-27632 at 37° C. under 5% CO₂. Cells from two intact corneas were seeded in one well of a 12-well plate.

The morphologies of the primary RPE cells and CECs were observed using an inverted contrast phase microscope (Nikon TE 2000-U, Nikon, Tokyo, Japan). Cells were subcultured when they reached confluence, and cultivated primary cells were used for the following transplantation.

### Example 2 (hESC Cell Line H1)

Based on the differentiation method priorly disclosed (Rapid Differentiation of Multi-Zone Ocular Cells from Human Induced Pluripotent Stem Cells and Generation of Corneal Epithelial and Endothelial Cells, Stem Cells Dev. 2019 Apr. 1; 28(7): 454-463), hESC cell line H1 cells were cultured till fusion rate reached approximately 80% using mTeSRI medium, then digested with 5 mg/ml type IV collagenase for 15 mins and seeded into 1% Matrigel-coated dish; cell differentiation medium 1 (DMEM/F12 and Neuralbasal medium (1:1), 2 mM glutamine, 0.1 mM non-essential amino acids, 0.1 mM β-mercaptoethanol and 1% N2 supplement) mixed with 2% Matrigel was utilized to culture for 2 days, then changed to Matrigel-free medium for 5 days; using differentiation medium 2 (DMEM/F12 medium, 10% serum substitute, 2 mM glutamine, 0.1 mM non-essential amino acid, 0.1 mM β-mercaptoethanol) to culture for 3 weeks; mechanical separation of retinal pigment epithelial cells and cell expansion.

### Example 3 (hiPSC Cell Line DY0100)

This example used the hiPSC cell line DY0100 to induce retinal pigment epithelial cells.

The induction differentiation method was the same as Example 1, using mTeSR1 medium to culture hiPSC cell line DY0100 till cell fusion reach approximately 80%, then using 5 mg/ml type IV collagenase to digest for 15 minutes, inoculating into 1% Matrigel-coated dishes, and using cell differentiation medium 1 mixed with 2% Matrigel to culture for 2 days, changing to Matrigel-free medium for 5 days; using differentiation medium 2 to culture for 3 weeks; mechanical separation of retinal pigment epithelial cells and cell expansion.

### Example 4 (Knockout of the Chromogenic Gene Tyrosinase)

In order to reduce pigmentation, the present example knocked out the chromogenic gene Tyrosinase, and prepared non-pigmented hESC/hiPSC-RPE cells, which can also maintain corneal transparency after transplantation. The present embodiment used CRISPR-Cas9 technology to specifically knock out the Tryosinase gene to prepare pigment-free retinal pigment epithelial cells.

In some embodiments, the hESC cell line H1 was used; In some other embodiments, the hiPS cell line DY0100 was used.

Construction of Tryosinase knockout cells: after digestion of the hESC cell line H1 or hiPSC cell line DY0100 which reached about 80% cell fusion, the cells were inoculated according to the ratio of 1:20˜30, and transfection reagent mixed with siRNA was added in the next day, transfection was performed when the confluence reached about 50-60% after 16-24 hours culture, and the amount of the added virus=(MOI×number of cells)/virus titers. After 12-20 hours of transfection, change to mTeSRI complete medium for 72-96 hours culture, and the transfection performance was evaluated according to fluorescence intensity. The sets with most fluorescent signals were selected for flow cytometry sorting, culture, and expansion to establish Tryosinase knockout cell lines.

Steps for induction of differentiation were the same as Example 2 or 3.

### Example 5 Intracameral Cell Transplantation and Postoperative Examinations

The rabbit corneal endothelial dysfunction model was performed according to our previously described scheme (Li Z, Duan H, Jia Y, Zhao C, Li W, Wang X, Gong Y, Dong C, et al. Long-term corneal recovery by simultaneous delivery of hPSC-derived corneal endothelial precursors and nicotinamide. J Clin Invest. 2022; 132(1): e146658. and Gong Y, Duan H, Wang X, Zhao C, Li W, Dong C, et al. Transplantation of human induced pluripotent stem cell-derived neural crest cells for corneal endothelial regeneration. Stem Cell Res Ther. 2021; 12(1): 214.). In short, rabbits were anesthetized with intramuscular ketamine hydrochloride (40 mg/kg, Gutian Pharmaceutical Co., Fujian, China) and intravenous pelltobarbitalum natricum (50 mg/kg, Sinopharm Chemical Reagent Co., Shanghai, China). The central corneal endothelium in the right eye of each rabbit was mechanically stripped from the DM using a 20-gauge soft silicone needle (Inami, Tokyo, Japan). Depending on the types of transplanted cells at the next stage, a curettage model of different sizes was adopted, a diameter of 7 mm was applied to primary RPE cells and CECs transplantation, and a diameter of 9 mm for hESC-derived RPE cells. Next, after irrigating the cell debris with saline, the surgeon injected heparin sodium (625 U/mL, Qianhong Bio-pharma Co., Changzhou, China) into the anterior chamber to reduce inflammation. Finally, the incision was sutured, and the model was established. The right eyes of corneal endothelial-damaged rabbits (n=3) without cell injection were used as a negative control, and the right eyes of normal rabbits (n=3) were used as a positive control.

Cells including primary rabbit RPE cells and CECs in Example 1-4, were dissociated with Accutase (StemCell Technologies, Vancouver, Canada) for 15 min at 37° C. and then gently triturated into cell suspension. Next, 3×10⁵˜1.2×10⁶ cells were dissolved in 200˜300 μl DMEM basal medium in addition of 10-100 μM Y27632 or/and 5-10 mM nicotinamide.For example, 3×10⁵ primary RPE cells, 3×10⁵ primary CECs, 5×10⁵-1×10⁶ hESC/hiPSC-derived RPE cells, and 8×10⁵ non-pigmented hESC/hiPSC-RPE cells RPE cells and in 250 μL DMEM supplemented with 100 μM Y-27632. Then the cell suspension was injected into the anterior chamber immediately after closing the incision. The rabbits were still under anesthesia after surgery and were kept in the eye-down position on the operating table for 3 h to promote cell attachment. For postoperative care, all operative eyes were treated with topical medication for 14 days. Specifically, tobramycin and dexamethasone eye drops (Novartis, Basel, Switzerland) with 10 mM Y-27632 were administered four times a day. Meanwhile, local subconjunctival injections of a 1:1 mixture of 5 mg/mL dexamethasone sodium phosphate (CISEN Pharmaceutical Co.) and 0.5 mg/mL atropine sulfate (Kingyork Co., Tianjin, China) were administered once daily.

Corneal clarity, central corneal thickness, and intraocular pressure of operative eyes on days 1, 3, 7, 14, and 30 were monitored and measured using slit-lamp microscopy (SL-D7, Topcon, Tokyo, Japan), optical coherence tomography (OCT, Fremont, USA), and a tonometer (Tono-Pen AVIA, Reichert, NY, USA), respectively.

### Results

### Comparison of Non-Pigmented Rabbit RPE Cells and CECs in Situ

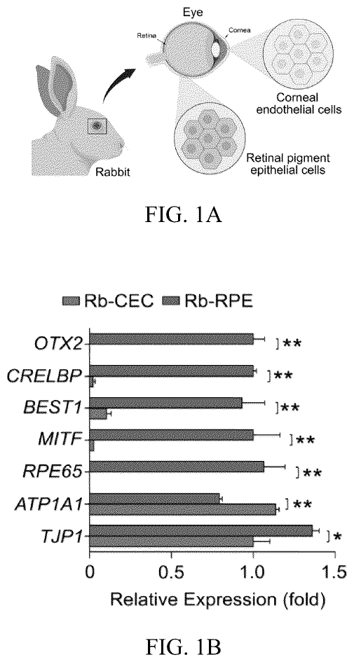

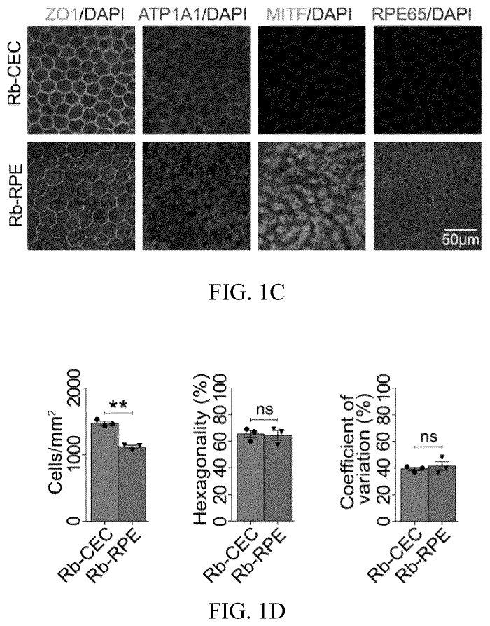

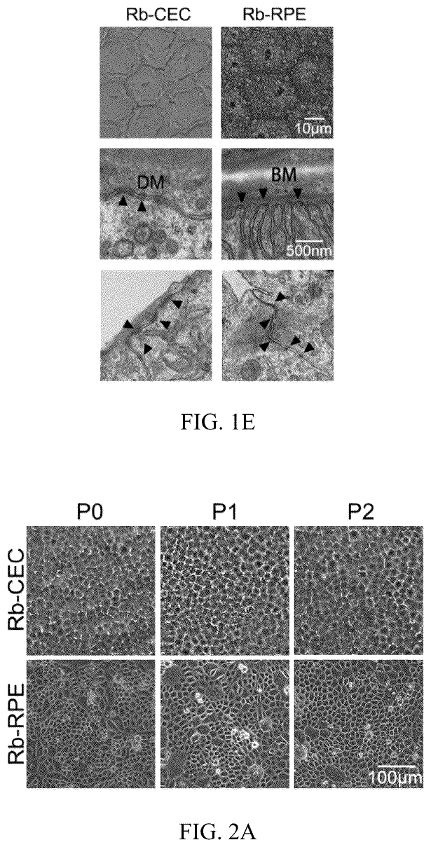

As is well known, both the corneal endothelium and retinal pigment epithelium (RPE) are monolayers of hexagonal cells involved in maintaining normal visual function (FIG. 1A). To evaluate the similarities and differences of RPE cells and CECs, we isolated and detected the in situ RPE and corneal endothelium. The analysis of qRT-PCR revealed that RPE cells expressed TJP1, ATP1A1, and RPE-related genes, including RPE65, MITF, BESTROPHIN, CRELBP, and OTX2, at mRNA levels. Compared with CECs, RPE cells exhibited higher TJP1 (P<0.05) and lower ATP1A1 (P<0.01) (FIG. 1B). Dual immunofluorescent staining showed that both RPE cells and CECs displayed tightly hexagonal intercellular borders and monolayer cellular morphologies of regular size and shape, accompanied by positive staining of ATP1A1 (FIG. 1C). Not surprisingly, RPE-related markers of RPE65 and MITF were positively expressed in RPE cells, compared to CECs (FIG. 1C). The statistical analysis, based on images of in situ ZO1 immunostaining, showed that there were no significant differences in hexagonality (P>0.05) and coefficient of variation (P>0.05), while the cell density of RPE cells was clearly less than that of CECs (P<0.01) (FIG. 1D).

To further verify the similarities and differences between RPE cells and CECs, we performed ultrastructural analyses of the RBCC and the CEC-DM complex, respectively. Scanning electron microscopy examinations revealed a similar monolayer, with continuous layers in vivo formed by the two types of hexagonal cells. Both RPE cells and CECs appeared healthy and in good shape, with well-defined cell boundaries and tightly opposed cell junctions. In addition, the apical surface of RPE cells was covered with microvilli (FIG. 1E). Transmission electron microscopy showed that CECs adhere to DM with hemidesmosome attachments, much as RPE cells attach to Bruch's membrane. Moreover, both CECs and RPE cells were tightly attached to neighboring cells by numerous desmosomes (FIG. 1E). Intracameral injection of cultured non-pigmented RPE cells and CECs Cultivation of primary non-pigmented rabbit RPE cells and CECs

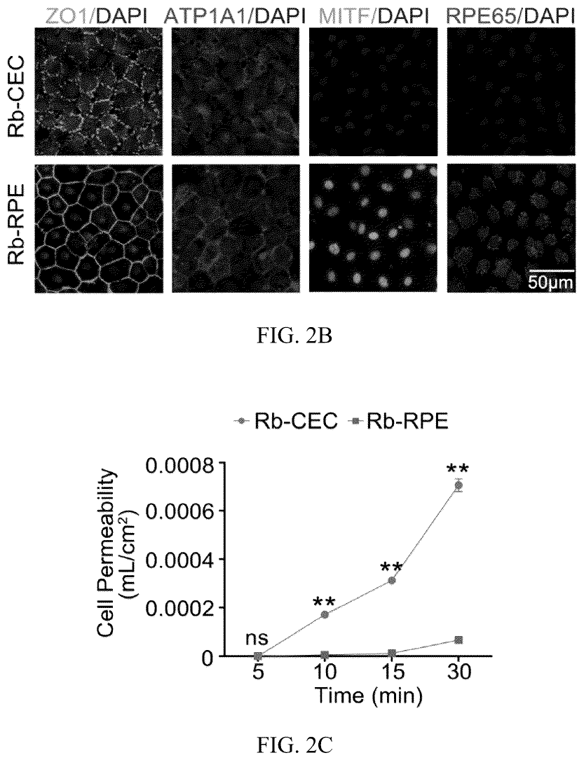

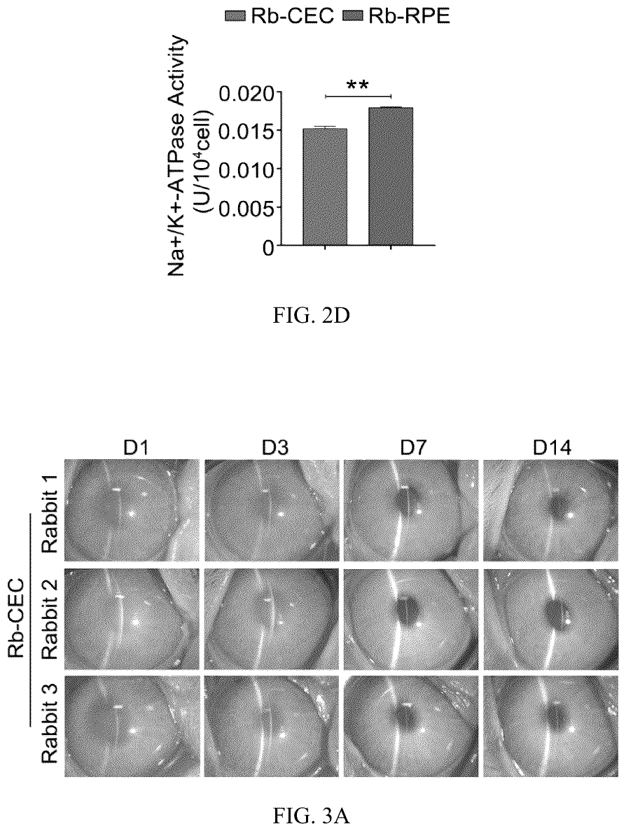

Primary non-pigmented rabbit RPE cells amplified, organized, and formed a tightly connected monolayer hexagonal structure during ex vivo culture, consistent with primary CECs (FIG. 2A). Furthermore, immunofluorescent staining showed positive expressions of ZO1 and ATP1A1 in primary RPE cells, as well as in CECs, while RPE cells were confirmed with positive staining of RPE65 and MITF (FIG. 2B). Since RPE cells have shown morphologically similarities to CECs, we tested the barrier functions and pumping functions of these two kinds of primary cells. As shown in FIG. 2C, the value of cell permeability in RPE cells was obviously weaker than that in CECs at 10 min, 15 min, and 30 min (P<0.01), while there was no significant difference at 5 min (P>0.05). Moreover, Na+, K+-ATPase activity of RPE cells was higher than that of CECs (P<0.01) (FIG. 2D). Intracameral injection of cultured non-pigmented RPE cells and CECs

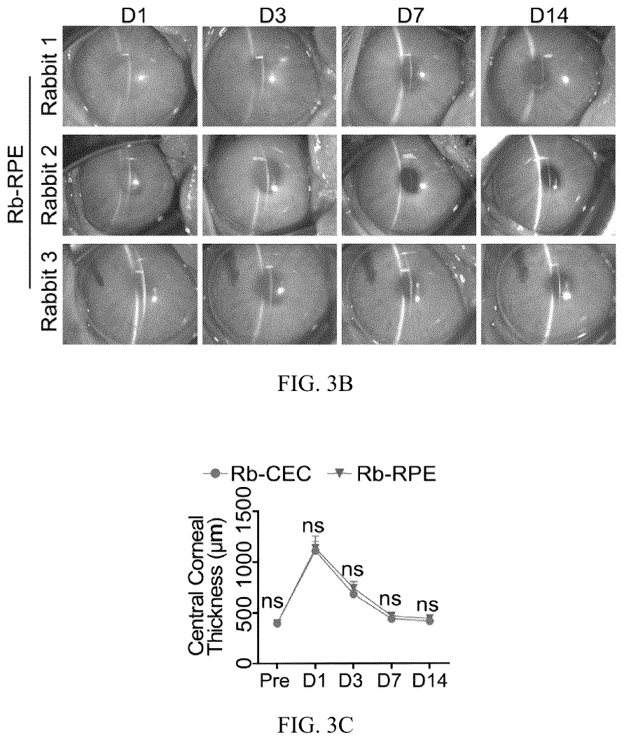

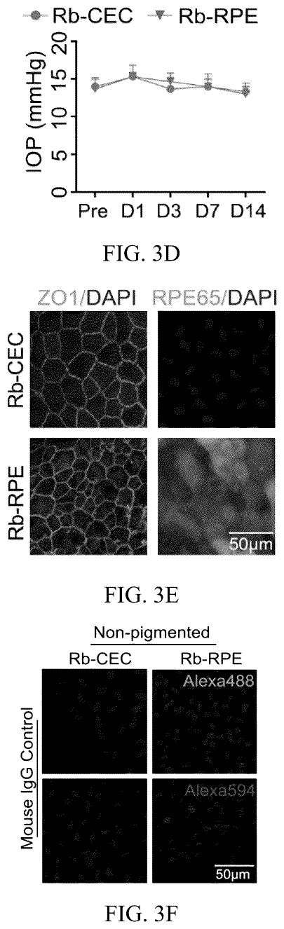

To evaluate the therapeutic effects of primary non-pigmented rabbit RPE cells on corneal endothelial dysfunction, a quantity of 3×10⁵ cells supplemented with 100 μM Y-27632 were injected into the anterior chamber of central corneal-scraped rabbit, with primary CECs as the control. Rabbits injected with RPE cells exhibited gradual corneal transparency after surgery, consistent with those injected with CECs (FIGS. 3A and 3B). Consistently, no significant difference in corneal central thickness was discovered between rabbits transplanted with non-pigmented RPE cells and CECs within 14 days after surgery (P>0.05) (FIG. 3C). Moreover, intraocular pressure was not increased by intracameral injection of either RPE cells or CECs (FIG. 3D).

To further determine the roles played by primary non-pigmented RPE cells in vivo, immunofluorescence staining with functional marker ZO1 and RPE-related marker RPE65 was performed on corneas with transplanted cells at day 14 postoperatively. Compared to CECs, transplanted RPE cells formed a similar regular distribution of ZO1, with positive staining of RPE65 (FIGS. 3E and FIG. 3F).

### Therapeutic Effects of Intracameral Injection of Primary Pigmented RPE Cells

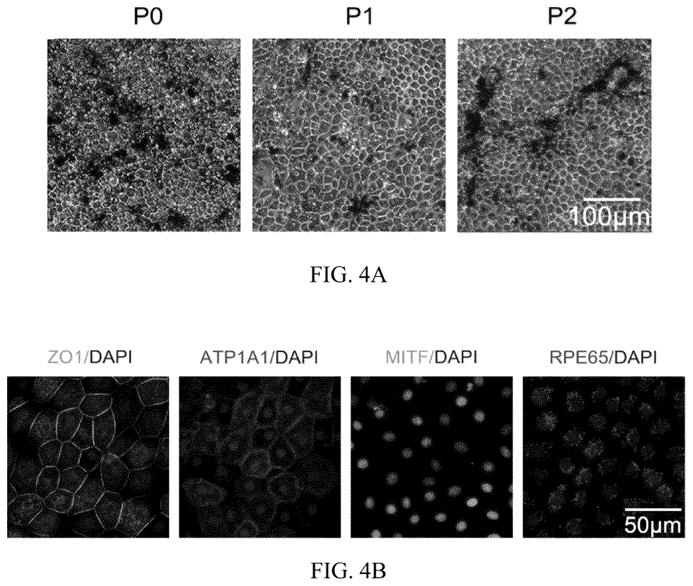

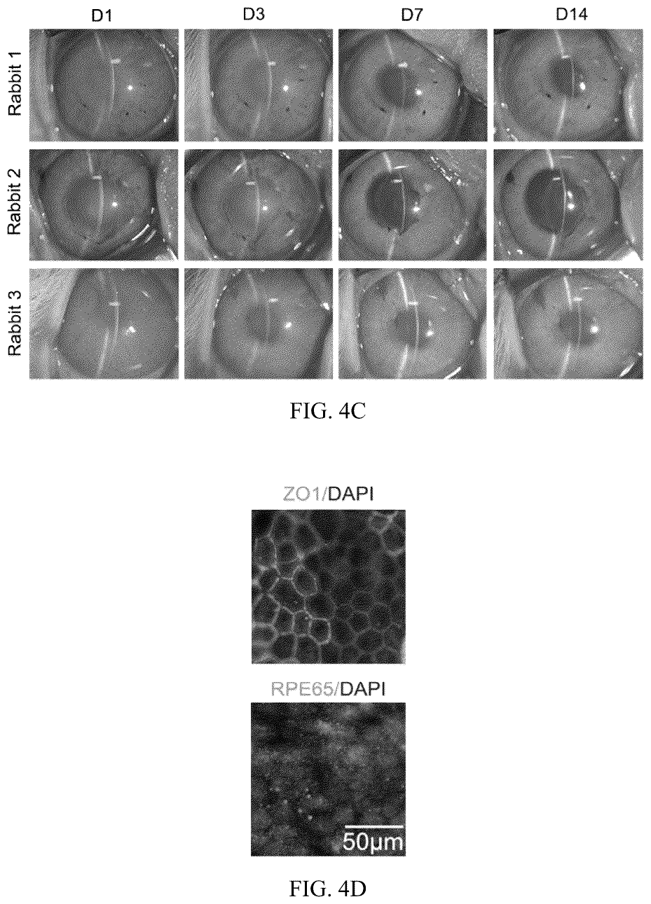

Human RPE cells contain melanin and appear dark brown. To verify the therapeutic effects of pigmented RPE cells for corneal endothelial dysfunction, primary pigmented RPE cells were derived and cultured, showing similar characteristics under the same culture conditions as non-pigmented RPE cells, except for substantial pigmentation (FIGS. 4A and 4B). Primary pigmented RPE cells were injected into the anterior chamber of the central corneal-scraped rabbits to estimate the effects for corneal endothelial dysfunction. The corneas with transplanted cells achieved a rapid improvement of corneal clarity and remained stable from day 7 after operation, accompanied by pigmentation on the corneal endothelium, exhibiting extensively distributed functional marker ZO1 and RPE-related marker RPE65 at day 14 postoperatively (FIGS. 4D and 4E).

Intracameral Injection of hESC/hiPSC-Derived RPE Cells

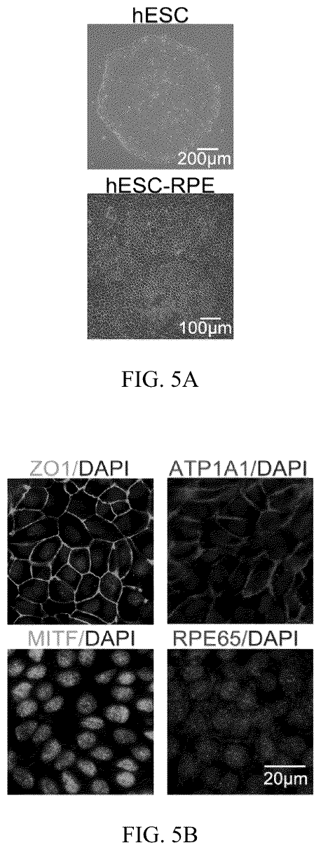

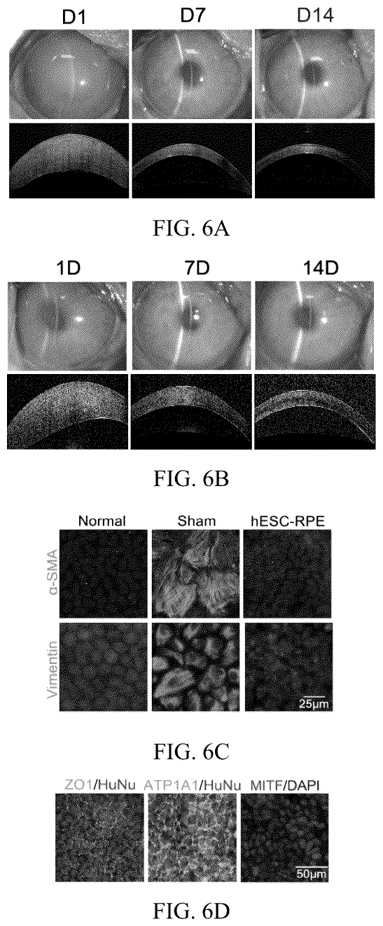

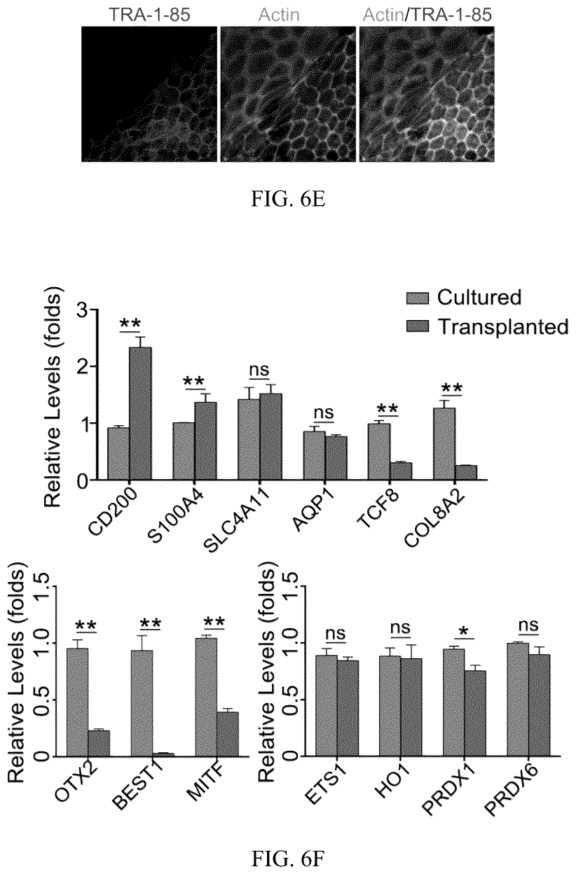

To further verify the availability of human RPE cells, we explored whether hESC/hiPSC-derived RPE cells could alleviate corneal endothelial dysfunction. As shown in FIGS. 5A and 5B, hESCs were induced into the polygonal RPE cells, which were positive staining of RPE-related markers MITF and RPE65, and exhibited regular and continuous distributions of ZO1 and ATP1A1 at the cell membrane. Then, 5×10⁵-1×10⁶ hESC/hiPSC-derived RPE cells were intracamerally injected into the rabbit model. The transplanted hESC/hiPSC-derived RPE cells rapidly improved corneal clarity and restored normal corneal thickness within 7 days (P>0.05) (FIG. 6A and 6B). Endothelial-mesenchymal transition (EnMT) is one of the major challenges of cell therapy to treat corneal endothelium damage. We demonstrated that transplanted cells in the central damaged arca were negative for the classical EnMT marker a-SMA and weakly positive for Vimentin 7 days after operation, which were obviously expressed in non-injected endothelial-damaged group (FIG. 6C). Next, immunostaining results also showed that transplanted cells were positive for human specific antibody human nuclei (HuNu), RPE-related marker MITF, and corneal endothelial functional markers ZO1 and ATP1A1 at day 14 postoperatively (FIG. 6D). Immunostainning of human specific antibodies TRA-1-85 showed the transplanted hESC-derived RPE cells survived in the corneal endothelium of the rabbits 14 days after transplantation, and Fibros actin (F-actin) staining revealed that the transplanted cells integrated with the host CECs (FIG. 6E). qRT-PCR analysis showed that relative expression of CEC markers such as CD200, S100A4 clearly increased in transplanted hESC-RPE cells at day 14 after surgery (P<0.01), accompanied by significant decreased expressions of specific RPE markers OTX2, BEST1, and MITF. However, some CEC-related functional genes such as SLC4A11 and AQP1 showed no significant changes, while the relative expression of TCF8 and COL8A2 decreased significantly postoperatively (P<0.01). Moreover, there were no significant changes in the expression levels of antioxidant-related genes, including ETS1, HO1, and PRDX6 (P>0.05) (FIG. 6F).

Intracameral Injection of Tryosinase Knockout hESC-Derived RPE Cells (Tyro KO-iRPE Cells) and Normal hESC-derived RPE Cells (iRPE)

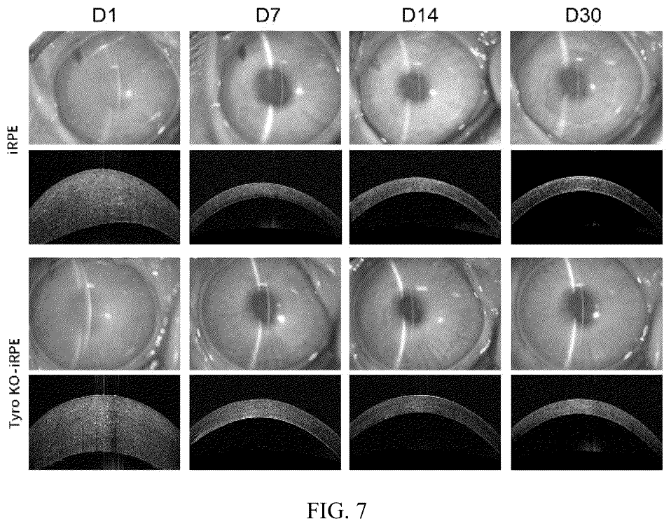

Tyro KO-iRPE cells were intracamerally injected into the rabbit model with iRPE cells as control. The transplanted Tyro KO-iRPE cells resolved corneal edema and decreased corneal thickness within 7 days. Importantly, the corneal transparency with Tyro KO-iRPE cells transplantation persisted to 1 month after operation, compared to iRPE cells transplanted rabbits with the light brown in central cornea. Moreover, there were no significant differences of corneal thickness between Tyro KO-iRPE cells and iRPE cells treated rabbits based on OCT images (FIG. 7).

Although the specific embodiments of the present invention have been described in detail, those skilled in the art would understand that according to all the teachings that have been disclosed, various modifications and substitutions may be made to those details and doses, which are within the scope of protection of the present invention. The full scope of the invention is given by the appended claims and any equivalents thereof.

1. A method for treating an eye disease in a subject in need thereof, comprising:
preparing a composition comprising retinal pigment epithelial cells; and
administering the composition to the anterior chamber of an eye of the subject.

2. The method according to claim 1, wherein the composition comprising a cell medium solvent, in which a ratio of the retinal pigment epithelial cells to a DMEM low-sugar culture medium is 3×10⁵-1.2×10⁶: 200-300 microliters.

3. The method according to claim 2, wherein the cell medium solvent is DMEM low-sugar culture medium.

4. The method according to claim 2, wherein the ratio of the retinal pigment epithelial cells to the cell medium solvent is 5×10⁵-1×10⁶: 200-300 microliters.

5. The method according to claim 1, further comprising one or more specific inhibitors selected from Y27632, nicotinamide, and TGF-β inhibitor SB431542 to the anterior chamber of the eye of the subject.

6. The method according to claim 5, wherein a dosage of Y27632 is 5-100 μM.

7. The method according to claim 1, wherein the subject suffers from corneal endothelial function decompensation or corneal endothelial dysfunction.

8. The method according to claim 1, wherein the retinal pigment epithelial cells are obtained by differentiating stem cells selected from human embryonic stem cells, human-induced pluripotent stem cells, primary culture of human, and rabbit retinal pigment epithelial cells.

9. The method according to claim 1, wherein the pigment-producing gene Tyrosinase in the retinal pigment epithelial cells is knocked out.

10. The method according to claim 1, wherein the pigment epithelial cells express corneal endothelial functional markers selected from ZO1 and ATP1A1.

11. The method according to claim 1, wherein the retinal pigment epithelial cells are obtained by differentiating stem cells selected from human embryonic stem cells or human-induced pluripotent stem cells.

12. A method for relieving or treating a corneal edema, a corneal thickness abnormality, or corneal transparency decline in a subject, which comprises administering to the subject a pharmaceutical composition comprising retinal pigment epithelial cells and a DMEM low-sugar culture medium, wherein the ratio of the retinal pigment epithelial cells to the DMEM low-sugar culture medium is 3×10⁵-1.2×10⁶: 200-300 microliters.

13. The method according to claim 12, wherein the ratio of the retinal pigment epithelial cells to the DMEM low-sugar culture medium is 5×10⁵-1×10⁶: 200-300 microliters.

14. The method according to claim 12, which further comprises administering to the subject one or more specific inhibitors selected from Y27632, nicotinamide, and TGF-β inhibitor SB43 1 542.

15. The method according to claim 1, wherein the subject has been diagnosed with a corneal thickness abnormality, corneal transparency decline, a corneal edema, a corneal endothelial injury, a corneal endothelial lesion, vision decline, vision loss, eye dryness, or eye pain.

16. The method according to claim 1, wherein the subject is a mammal.
